(19) United States
(12) Patent Application Publication
(10) Pub. No.: US 2018/0168549 A1
(43) Pub. Date: Jun. 21, 2018

(54) ULTRASONIC SCANNER WITH A MULTIPLE FACETED MIRROR

(71) Applicant: Biim Ultrasound AS, Narvik (NO)

(72) Inventor: Trygve Burchardt, Howell, MI (US)

(21) Appl. No.: 15/894,663

(22) Filed: Feb. 12, 2018

Related U.S. Application Data
(63) Continuation of application No. PCT/IB2016/054876, filed on Aug. 12, 2016.
(60) Provisional application No. 62/205,274, filed on Aug. 14, 2015.

(51) Int. Cl. A61B 8/00; G01N 29/22; G01N 29/26; A61B 8/14; G10K 11/20

(52) U.S. Cl. CPC A61B 8/4461; G01N 29/221; G01N 29/262; A61B 8/4494; G10K 11/20; A61B 8/54; A61B 8/4427; A61B 8/145; A61B 8/56

(57) ABSTRACT

An illustrative device for creating images based on ultrasonic pulses comprises an ultrasonic transducer configured to transmit and receive ultrasonic pulses; and a mirror comprising at least two facets configured to reflect the ultrasonic pulses, wherein the mirror is configured to rotate. The ultrasonic transducer and the mirror are positioned in a probe head. A first facet of the mirror reflects the ultrasonic pulses from the ultrasonic transducer during a first range of rotation of the mirror and a second facet of the mirror reflects the ultrasonic pulses from the ultrasonic transducer during a second range of rotation of the mirror.

### CROSS-REFERENCE TO RELATED APPLICATIONS

This application claims priority to and the benefit of U.S. Provisional Patent Application No. 62/205,274, filed Aug. 14, 2015, which is incorporated herein by reference in its entirety.

### BACKGROUND

This disclosure relates to methods and apparatuses for imaging sections of a body, (e.g., a human body) by transmitting ultrasonic energy into the body and determining the characteristics of the ultrasonic energy reflected therefrom. More particularly, this disclosure relates to an improved ultrasonic scanning technique and system with an acoustic mirror having multiple facets.

The following description is provided to assist the understanding of the reader. None of the information provided or references cited is admitted to be prior art. Some ultrasonic imaging devices include a transducer and a rotating acoustic mirror. However, there may be limitations or trade-offs between frame rate and quality of the ultrasound image with this design. Thus, it is desirable to overcome these trade-offs to maintain, or even improve, frame rate or quality while also improving the other.

### SUMMARY

An illustrative device for creating images based on ultrasonic pulses comprises an ultrasonic transducer configured to transmit and receive ultrasonic pulses; and a mirror comprising at least two facets configured to reflect the ultrasonic pulses, wherein the mirror is configured to rotate. The ultrasonic transducer and the mirror are positioned in a probe head. In some embodiments, the mirror is configured to reflect the ultrasonic pulses while rotating, and wherein received ultrasonic pulses are used to determine an ultrasonic scan of material adjacent to the lens.

In some embodiments, a first facet of the mirror is configured to reflect the ultrasonic pulses from the ultrasonic transducer during a first range of rotation of the mirror and a second facet of the mirror is configured to reflect the ultrasonic pulses from the ultrasonic transducer during a second range of rotation of the mirror. In some embodiments, the device is configured such that the ultrasonic pulses received at the transducer reflected from the first facet are used to determine a first ultrasonic scan of material adjacent to the lens, and wherein the device is configured such that the ultrasonic pulses received at the transducer reflected from the second facet are used to determine a second ultrasonic scan of material adjacent to the lens.

In some embodiments, the device is configured such that the first and second ultrasonic scans determined from one rotation of the mirror increase a frame rate of a resultant ultrasonic image without diminishing quality of the resultant ultrasonic image. In some embodiments, the device is configured such that the first and second ultrasonic scans determined from one rotation of the mirror increase the image quality of a resultant ultrasonic image without diminishing a frame rate of the resultant ultrasonic image.

In some embodiments, the probe head further comprises a lens configured to direct the ultrasonic pulses. In some embodiments, the mirror is configured to rotate at a rotational speed of the motor. In some embodiments, the ultrasonic transducer is a circular transducer with rings for beam forming.

In some embodiments, the mirror has at least one additional facet. In some embodiments, the mirror is configured in a pyramid shape. In some embodiments, the mirror is configured in a double-pyramid shape, wherein a first pyramid has at least two facets and a second pyramid has at least two facets, and wherein a base of the first pyramid is attached to a base of the second pyramid.

In some embodiments, the device further comprises a second ultrasonic transducer disposed in the probe head on the opposing side of the mirror from the first ultrasonic transducer. In some embodiments, the ultrasonic transducer is configured to transmit ultrasonic pulses at a first frequency and the second ultrasonic transducer is configured to transmit ultrasonic pulses at a second frequency that is different than the first frequency. In some embodiments, the device is configured such that ultrasonic pulses received at the first or the second transducer from the transmission of pulses at a first frequency are combined with the ultrasonic pulses received at the first or second transducer from the transmission of pulses at a second frequency to determine an ultrasonic scan of material adjacent to the lens. In some embodiments, the first frequency is in the range of 1 to 5 MHz and the second frequency is in the range of 8 to 20 MHz.

In some embodiments, the device further comprises an electronics chamber that comprises a battery and electrical circuitry, wherein the electrical circuitry is electrically connected to the ultrasonic transducer. In some embodiments, the electrical circuitry is configured to control an operational parameter of the device or perform a function on the received ultrasonic pulses.

In some embodiments, controlling the operational parameter of the device comprises controlling at least one operational parameters selected from the group consisting of: (a) the frequency of pulses generated by the transducer, (b) the pulse length of pulses generated by the transducer, (c) the timing of pulses generated by the transducer, and (d) the number of pulses generated by the transducer. In some embodiments, the function performed on the received ultrasonic pulses is a function selected from the group consisting of: (a) registering the voltage variations on the transducer, (b) converting the signal to digital, (c) performing beam forming upon reception of the signals, (d) performing scan conversion, (e) performing demodulation, and (f) performing variable gain compensation.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF DRAWINGS

FIG. 1A is a top perspective view of an ultrasound probe with an acoustic mirror in accordance with an illustrative embodiment.

FIG. 1B is a side view of an ultrasound probe with an acoustic mirror in accordance with an illustrative embodiment.

FIG. 1C is a front end view of an ultrasound probe with an acoustic mirror in accordance with an illustrative embodiment.

FIG. 2A is a top perspective view of an ultrasound probe head with a multi-faceted acoustic mirror in accordance with an illustrative embodiment.

FIG. 2B is a side view of an ultrasound probe head with a multi-faceted acoustic mirror in accordance with an illustrative embodiment.

FIG. 3A illustrates an alternative configuration of a multi-faceted acoustic mirror in accordance with an illustrative embodiment.

FIG. 3B illustrates the multi-faceted acoustic mirror of FIG. 3A in accordance with an illustrative embodiment.

FIG. 4A illustrates an alternative configuration of a multi-faceted acoustic mirror in accordance with an illustrative embodiment.

FIG. 4B illustrates the multi-faceted acoustic mirror of FIG. 4A in accordance with an illustrative embodiment.

FIG. 5 is a top perspective view of an ultrasound probe with the multi-faceted acoustic mirror of FIG. 4A in accordance with an illustrative embodiment.

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Ultrasound imaging techniques are often used in clinical diagnostics. Ultrasound differs from other forms of radiation used for imaging in its interaction with living systems in that ultrasound is a mechanical wave. Accordingly, the information provided by the use of ultrasonic waves is of a different nature than that obtained by other methods and is found to be complementary to other diagnostic methods, such as those employing X-rays. Also, the risk of tissue damage using ultrasound appears to be much less than the apparent risk associated with ionizing radiations such as X-rays. Ultrasound imaging devices can be used in settings other than a medical setting. For example, ultrasound imaging devices can be used for finding cracks in materials (e.g., metals, steel, etc.), diagnosing machinery malfunctions, etc.

Many diagnostic techniques using ultrasound are based on a pulse-echo method, wherein pulses of ultrasonic energy are periodically generated by a suitable piezoelectric transducer. Each short pulse of ultrasonic energy is focused to a narrow beam, which is transmitted into the patient's body in which the energy eventually encounters interfaces. Once the energy encounters interference, a portion of the ultrasonic energy is reflected at the boundary back to the transducer. After generation of the pulse, the transducer operates in a “listening” mode in which the device converts received reflected energy, or “echoes,” from the body into electrical signals. The time of arrival of the echoes depends on the distance of the interfaces from the device and the propagation velocity of the ultrasonic energy. The amplitude of the echoes is indicative of the reflection properties of the interphase and, accordingly, of the nature of the characteristic structure forming the interphase. In alternative embodiments, any suitable method of transmitting and/or receiving ultrasonic energy may be used.

There are various ways in which the information in the received echoes can be usefully presented. One common form of display is referred to as a “B-scan.” In a B-scan, the echo information is of a form similar to a conventional television display. That is, the received echo signals are utilized to modulate the brightness of the display at each point scanned. This type of display is useful, for example, when the ultrasonic energy is scanned transverse to the body so that individual “ranging” information yields individual scan lines on the display, and successive transverse positions are utilized to obtain successive scan lines on the display. The two-dimensional B-scan technique yields a cross-sectional picture in the plane of the scan, and the resultant display can be viewed directly and/or be recorded. In most instances, ultrasonic energy is almost totally reflective at interfaces with gas. Thus, coupling fluid, such as water or oil, or a direct-contact type transducer can be used to limit the amount of gas through which the ultrasonic energy passes.

An illustrative type of apparatus having a console includes a timing signal generator, energizing and receiving circuitry, and a display/recorder for displaying and/or recording image-representative electronic signals, such as those described in U.S. Pat. No. 4,084,582 and U.S. Pat. No. 6,712,765. A portable scanning head or module, suitable for being hand held, can have a fluid-tight enclosure having a scanning window formed of a flexible material. A transducer in the portable scanning module converts energy and also converts received ultrasound echoes into electrical signals, which are coupled to the receiver circuitry. A focusing lens is coupled to the transducer, and a fluid, such as water or oil, fills the portable scanning module in the region between the focusing lens and the scanning window. A reflective scanning mirror is disposed in the fluid, and a driving motor, energized in synchronism with the timing signals, drives the scanning mirror in a periodic fashion. The ultrasonic beam is reflected off of the scanning mirror and into the body being examined via a scanning window formed of a rigid material.

For a two dimensional B-scan taken with the above-described type of scanning head, the dimensions scanned are: (1) depth into the body, which varies during each display scan line by virtue of the ultrasonic beam travelling deeper into the body as time passes; and (2) a slower transverse scan caused by the scanning mirror. The display is typically in a rectangular format (for example, the familiar television type of display with linear sweeps in both directions). However, the transverse scan of the ultrasonic beam itself, as implemented by the scanning mirror, results in a sector scan. For distances deeper in the body, the fanning out of the sectors results in geometrical distortion when displayed on a linear rectangular display. For example, the azimuth dimension in the extreme far field may be, for example, 2.5 times larger than the azimuth dimension in the extreme near field. Thus, the density of information on the far field side of the display is much higher than the density of information on the near field side of the display. In other words, what appears to be equal distance in the body on the far field side and the near field side of the display are actually different distances.

In some embodiments, the scanning window is in the form of an acoustic lens for converging the scan of the ultrasonic beam incident thereon that forms within the enclosure, as in U.S. Pat. No. 4,325,381. In some embodiments, the acoustic lens reduces geometric distortion of the scan of the ultrasonic beam. In an embodiment, the window/lens is formed of a rigid plastic material in a substantially plano-concave shape, with the concave surface facing the outside of the enclosure. In such an embodiment, the window/lens is provided with a focal length of about 1.5 times the distance between the reflective scanning means and the windows/lens and may be particularly suitable for a functioning embodiment. In alternative embodiments, any suitable lens, window, focal length, etc. may be used.

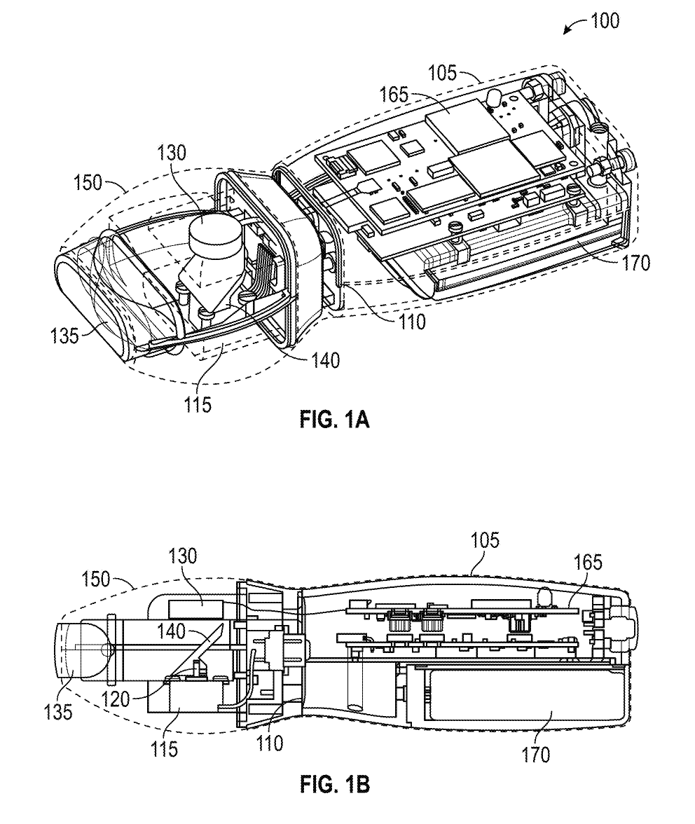

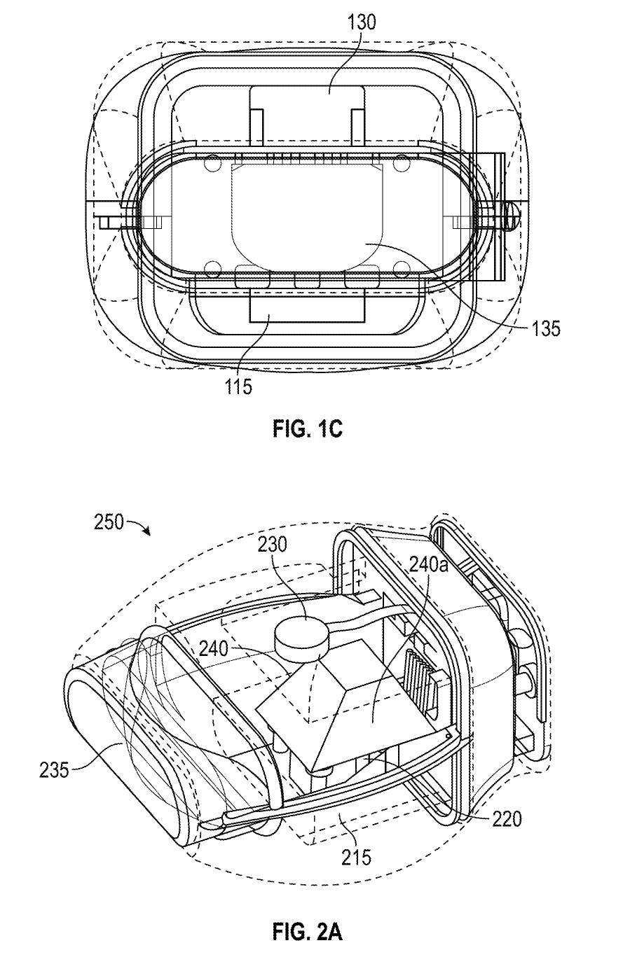

FIGS. 1A-1C depict an ultrasound probe 100 using a circular transducer 130 and a rotating acoustic mirror 140 to position the ultrasound beam and enable scanning functions. Compared to the more traditional linear phase array transducers, improved focus of the ultrasound pulse can be obtained from this configuration. FIG. 1A is a diagram of an ultrasound probe from a top view in accordance with an illustrative embodiment. FIG. 1B is a diagram of an ultrasound probe from a side view in accordance with an illustrative embodiment. FIG. 1C is diagram of a front end view of an ultrasound probe in accordance with an illustrative embodiment. In alternative embodiments, additional, fewer, and/or different elements may be used. An ultrasound probe 100 includes an electronics chamber 105 containing circuitry 165 and a battery 170, a wall 110, a motor 115, a motor shaft 120, a transducer 130, a lens 135, a mirror 140, and a probe head 150.

The electronics chamber 105 houses electronics and possibly batteries and is attached to the probe head 150. In some embodiments, the chamber 105 is removably attached to the probe head 150. The probe head 150 includes the motor 115 that is attached to the mirror 140. The angle of the mirror 140 and the location of the transducer 130 are arranged, such that the propagation pathway of the ultrasonic pulses is through the lens 135, is reflected by the mirror 140, and is received by the transducer 130, or vice versa after the pulse is transmitted by the transducer 130. In the embodiment illustrated in FIGS. 1A and 1B, the plane of the lens 135 is substantially perpendicular to the transducer 130 and the angle of the mirror is 45°. In other embodiments, alternative configurations for the plan of the lens 135 and the angle of the mirror 140 may be used. In some embodiments, the output shaft of the mirror is mechanically coupled to the mirror. In other embodiments, the mirror may be indirectly coupled to the motor.

In order to reduce attenuation of the ultrasound wave, the probe head 150 (including a cavity in the probe head) can be liquid filled. The liquid can be any suitable liquid such as water or oil. A cavity may be positioned on the opposite side of the mirror 140 from the transducer 130.

The motor 115 is connected to the mirror 140 via a shaft and when the motor is running, the mirror 140 rotates. In the illustrations shown in FIGS. 1A-1C, the rotating mirror is in the correct positioning to the transducer 130 to reflect the pulses about ⅙ of its rotation time. When the mirror is correctly positioned to the transducer, ultrasound pulses will be reflected from the mirror and out of the probe head through the lens 135. By using a time delay between rings on the transducer 130, beam forming of the pulse is possible. The beam forming electronics controls this, which can be programmed to give the correct focus depth for the ultrasound pulse in the body.

From the transducer, the ultrasound pulses travel through the probe chamber, which may be liquid filled, is reflected by the mirror and travels out through the probe head. When a lens is placed at the probe head 150 a linear image is produced. The rotation of the mirror results in each pulse to be reflected at a different angle. This creates the scanning function. The pulse duration is typically only a few microseconds, such as between 0.5 ms and 10 ms. After a pulse has been fired, the transducer goes into “listening” mode, at which time it then acts as a receiver for the echo ultrasound signals. The time from sending a pulse to receiving an echo back is typically a few hundred microseconds. The rotational speed of the motor determines how many pulses can be fired with each rotation, as the mirror is in position only part of the time.

As the motor 115 spins, the mirror 140 spins, thereby altering the path of the ultrasonic pulses emitted by the transducer 130 and transmitted through the lens 135. The altered path allows the ultrasound probe 100 to scan the medium at the end of the ultrasound probe 100 (e.g., a human body) without moving the transducer. Each time the motor passes the mirror, one 2D picture can be captured. The frame rate for ultrasound in video mode is, thereby, determined by the rotational speed. With increased rotational speed of the motor, the frame rate increases for the imaging, giving better video functionality. However, with a faster rotation of the motor fewer pulses can be fired as the mirror face passes the transducer, reducing the resolution of the picture captured.

A compromise between frame rate, resolutions and depth of penetration therefore has to be made. For example, rotating the mirror at 600 rpm gives a frame rate of 10 pictures per second. If the distance to and from the transducer and into the body is 6 cm, the speed of the ultrasound wave on average is 1540 m/sec, and the mirror is 3 cm wide., the number of 0.75 ms pulses is calculated to be 120 per rotation. If the rotation speed is increased to 1200 rpm (giving video of 20 frames/second) on the same configuration, the number of pulses is reduced to 60, thereby lowering the quality while increasing the frame rate. It is preferred to have as high frame rate and as many lines (pulses) on the image as possible. Typically, the difference in the image quality is mirror with increased pulses above 120. Therefore, it would be desirable to maintain this image quality (120 pulses per rotation) at a faster frame rate. Generally, ultrasound equipment runs at 10 to 40 frames per second.

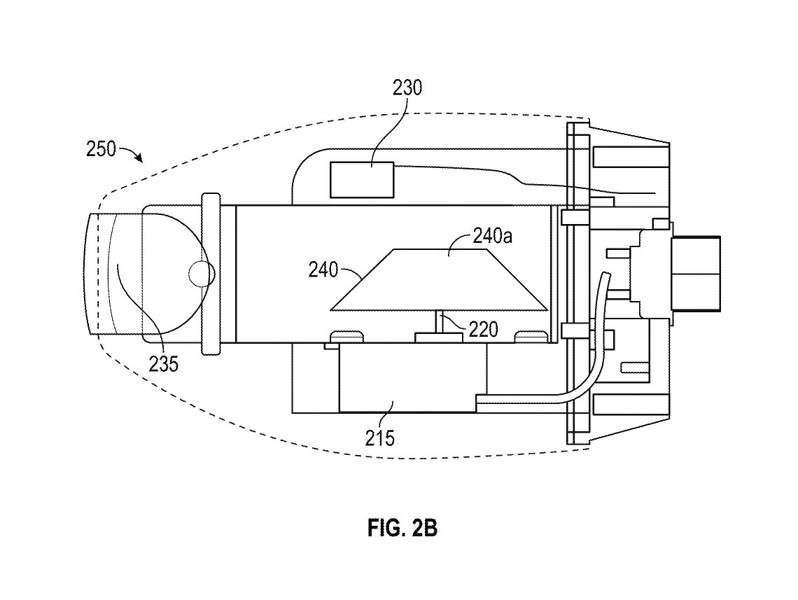

FIG. 2A is a diagram of an ultrasound probe head with a multi-faceted mirror from a top perspective view in accordance with an illustrative embodiment. FIG. 2B is a diagram of an ultrasound probe head with a multi-faceted mirror from a side view in accordance with an illustrative embodiment. FIG. 2C is a diagram of an ultrasound probe head with a multi-faceted mirror from a front end view in accordance with an illustrative embodiment. In alternative embodiments, additional, fewer, and/or different elements may be used. An ultrasound probe head 250 includes a motor 215, a motor shaft 220, a transducer 230, a lens 235, and a mirror 240.

The various elements of ultrasound probe head 250 are similar to the elements of the ultrasound probe 100 of FIGS. 1A-1C, and may be similarly configured, except that the mirror 240 has been configured in a pyramid-shape with four facets (such as facet 240a) that can reflect the ultrasound beam as it passes the transducer. As the motor 215 spins, the mirror 240 spins, thereby altering the path of the ultrasonic pulses emitted by the transducer 230 and transmitted through the lens 235. The altered path allows the ultrasound probe having probe head 250 to scan the medium at the end of the ultrasound probe (e.g., a human body) without moving the transducer.

In this configuration with a mirror having four facets, four images can be generated from each rotation of the mirror. Therefore, the rate of rotation can be slowed while maintaining the same preferred frame rate. As such, the time that the transducer is correctly aligned to the mirror is increased. The benefit of increasing the exposure time is that more pulses can be fired and reflected, and therefore, the pixel resolution of the image is improved.

Another advantage of a multi-faceted mirror is that the plurality of facets can enable different regions of beam focusing to be generated from the transducer during each rotation. For example, with four facets, four pictures are created with each rotation. The beam forming may be altered between them so that they have different focus points. By superimposing the multiple images on top of each other, a picture with a larger focus area is produced. More particularly, the beam forming shapes the ultrasound wave so that a focal point is formed at a given distance. Ray tracing models of the beam path enable accurate calculations of the focal point. Including this model into an algorithm allows for the focal point to be adjusted to penetrate deeper or shallower into the body. With multiple images produced per revolution, it is contemplated that this focal point could be adjusted from one image to the next, and the images compounded. This then produces an image with better resolution over a larger depth of penetration, without sacrificing frame rates.

The probe head 250 may be coupled to a prove body having an electronics chamber (similar to electronics chamber 105 of probe 100). The electronics chamber houses electronics and possibly batteries and is attached to the probe head 250. In some embodiments, the chamber is removably attached to the probe head 250. The electronics incorporates standard ultrasound processors. On the transmit side, the electronics controls the frequency, the pulse length, the timing and the number of pulses generated by the transducer. The circuitry also control the beam forming during transmission by setting the timing for the various rings on the transducer. On the receive side of the electronics, the electronics register the voltage variations on the transducer, converts the signal to digital, performs beam forming upon reception of the signals, scan conversion, demodulation, variable gain compensation and other classical ultrasound signal processing methods. The data may then be sent to an external devices, such as via wifi to a tablet for further post processing and image display.

The electronics also controls the rotation of the mirror and the timing for positioning of the mirror so that the mirror, transducer and lens alignment is optimal for the sending and receiving of the ultrasound signals. Battery control and monitoring may also be controlled by the electronics.

The probe head 250 includes the motor 215 that is attached to the mirror 240. The angle of the mirror 240 and the location of the transducer 230 are arranged such that the propagation pathway of the ultrasonic pulses is through the lens 235, is reflected by the mirror 240, and is received by the transducer 230, or vice versa after the pulse is transmitted by the transducer 230. In the embodiment illustrated in FIGS. 2A and 2B, the plane of the lens 235 is substantially perpendicular to the transducer 230 and the angle of each facet of the mirror is 45°. In other embodiments, alternative configurations for the plan of the lens 235 and the angle of the facets of the mirror 240 may be used.

In order to reduce attenuation of the ultrasound wave, the probe head 250 (including a cavity) can be liquid filled. The liquid can be any suitable liquid such as water or oil. A cavity may be positioned on the opposite side of the mirror 240 from the transducer 230.

The motor 215 is connected to the mirror 240 via a shaft and when the motor is running, the mirror 240 rotates. In the illustrations shown in FIGS. 2A-2C, the mirror shaft, or axis of rotation, is not centered with the transducer 230. In this way, the beam formed by the transducer is correctly aligned with the facets of the mirror 240 as the mirror rotates. When the mirror is correctly positioned to the transducer, ultrasound pulses will be reflected from the mirror facet and out of the probe head through the lens 235. As with the embodiment of FIGS. 1a-1C, by using a time delay between rings on the transducer 230, beam forming of the pulse is possible. The beam forming electronics controls this, which can be programmed to give the correct focus depth for the ultrasound pulse in the body.

When a lens is placed at the probe head 250 a linear image is produced. The rotation of the mirror results in each pulse to be reflected at a different angle. This creates the scanning function. The pulse duration is typically only a few microseconds, such as between 0.5 ms and 10 ms. After a pulse has been fired, the transducer goes into “listening” mode, at which time it then acts as a receiver for the echo ultrasound signals. The time from sending a pulse to receiving an echo back is typically a few hundred microseconds. The rotational speed of the motor determines how many pulses can be fired with each rotation, as the mirror is in position only part of the time.

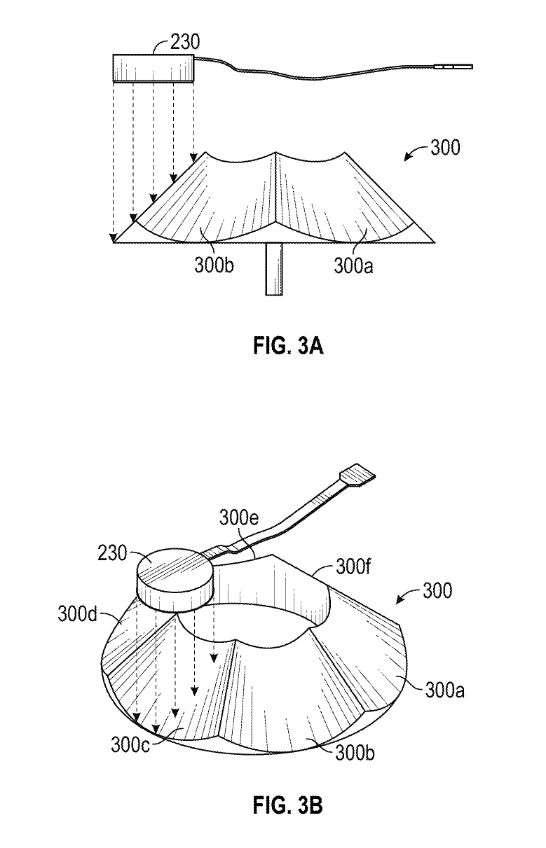

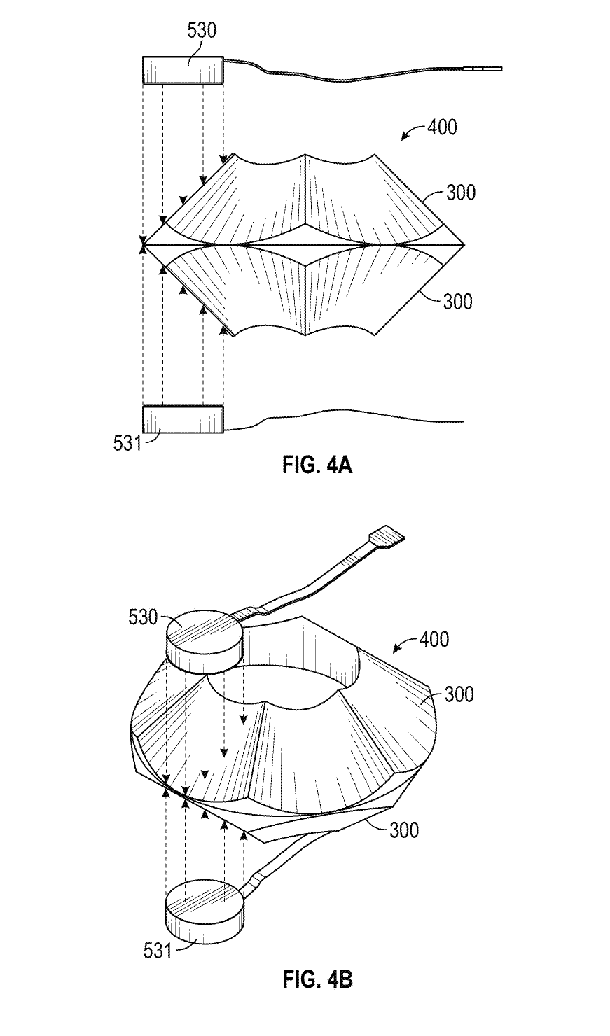

Though the embodiment illustrated in FIGS. 2A-2C shows a mirror having four facets, it is understood that the mirror may have any number of facets, including but not limited to 2, 3, 5, 6, 7, 8, 9, or 10 facets. As specific examples, FIGS. 3A-4B are illustrations of various configurations of multi-faceted mirrors that may be used in an ultrasound probe according to the present disclosure.

As shown in FIGS. 3A and 3B, a mirror 300 may have six facets 300a-300f arranged about the circumference of the mirror body. In the illustrative embodiment shown, the six facets are equally sized and shaped, though other configurations wherein the facets are unequal may also be used. In certain preferred embodiments, the mirror facets are cut at a 45° angle. A six-faceted mirror, such as mirror 300, will multiply the number of frames per rotation, or frames per second, six fold at the same line density and rotational speed of the motor (therefore, maintaining image pixel quality). In particular, the frames per second may increase from 10 to 60. The mirror itself may shaped as a large pyramid, or a pyramid with its top cut off. As shown in the illustration, the axis of rotation is centered, or goes through the tip of the mirror. However, also as shown, the axis is skewed away from the transducer center. In certain preferred embodiments, the size of the mirror, and more particularly the size of each individual facet, is sufficiently large so that the maximum diameter of the beam from the transducer is fully reflected off the mirror. The more facets cut in the mirror, the larger the mirror becomes in order for each facet to reflect the maximum diameter of the beam from the transducer. Alternatively, the diameter of the transducer may be reduced.

Alternatively, instead of capturing six-times the number of scans for each rotation, the mirror facets can be arranged to change the focal point on transmission of the pulses to six locations. This can provide for better resolution in an image where the six obtained scans are combined.

As shown in FIGS. 4A and 4B, a mirror 400 could be configured as a double-pyramid, such as two oppositely disposed six-faceted mirrors 300. With this mirror configuration, the probe would incorporate a second transducer 531 positioned opposite the mirror from the first transducer 530. This second transducer 531 could e.g. have a different focal zone or different frequency. As with the illustrative embodiment of FIGS. 3A-3B, the double pyramid mirror also rotates about an axis that is off-center from a transducer axis. In this probe, there may also be a new lens which has two inner parts which steers the beam from each transducer towards the center of the outer-beam to make the pulse from each transducer align in-plane for imaging.

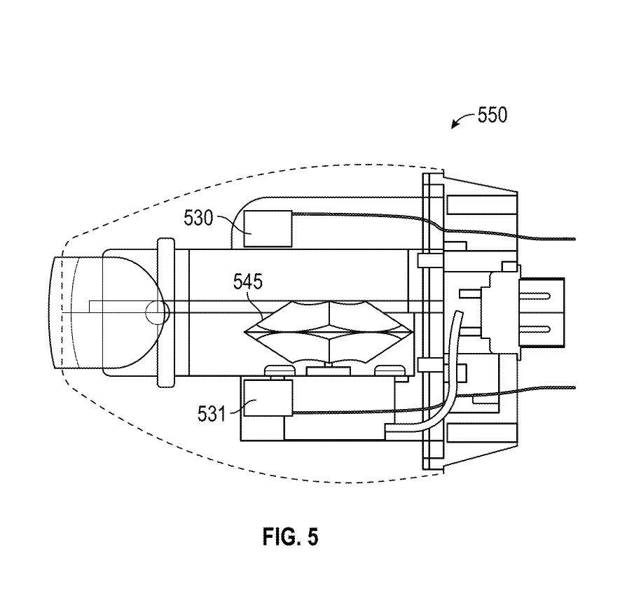

FIG. 5 is a diagram of another illustrative example of an ultrasound probe head 550 with the multi-faceted mirror, particularly the double six-faceted mirror 400 of FIGS. 4A and 4B. An ultrasound probe head 550 includes a motor 515, a first transducer 530, a second transducer 531, and a mirror 545.

The ultrasound probe head 550 has similar elements as the ultrasound probe head 250 of FIGS. 2A-2C, except that the probe head 550 includes a mirror having twelve total facets, six on each side of the mirror 545.

FIG. 5 also shows an embodiment wherein the ultrasound probe head 550 has a second transducer 531. With the use of two transducers, the time in which the mirror is in the correct position for the transducer to transmit a pulse is doubled and, thereby, the image quality can be improved at a given frame rate or, alternatively, the frame rate can be improved at a given image quality.

In an alternative use, the two transducers have different frequency ranges. Conventional transducers are built with a specific frequency range. Typically for ultrasound imaging for medical applications, the frequency is in the 1 to 20 MHz range. The frequency of the ultrasound pulse from the transducer is set according to the depth of penetration required. For imaging of organs deep into the body a low frequency is used. For imaging close to the skin a high frequency is used. The higher the frequency, the lower the wavelength so better resolution. However, high frequency attenuates more easily than low frequency, thus for deeper imaging the echo becomes weak making it difficult to distinguish it from noise. Therefore, lower image quality is obtained when using high frequency for deep imaging.

The use of two or more transducers in the ultrasound probe with different frequency range allow for both shallow and deep penetration imaging. It is possible to set, for instance, one transducer in the 1 to 5 MHz range and a second one in the 8 to 20 MHz range. This may allow users to do more investigations without requiring a variety of probes. It can reduce cost and improving quality and flexibility.

In other embodiments, more than two transducers may be used, if needed. As discussed above, the type and number of transducers is determined by evaluating the trade-offs between number of pulses needed with each pass of the mirror, the frame rate required for video display, the number of focus points on the image and the depth profile required.

In an illustrative embodiment, any of the operations described herein can be implemented at least in part as computer-readable instructions stored on a computer-readable memory. Upon execution of the computer-readable instructions by a processor, the computer-readable instructions can cause a node to perform the operations.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, unless otherwise noted, the use of the words “approximate,” “about,” “around,” “substantially,” etc., mean plus or minus ten percent.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A device for creating images based on ultrasonic pulses, the device comprising:
an ultrasonic transducer configured to transmit and receive ultrasonic pulses; and
a mirror comprising at least two facets configured to reflect the ultrasonic pulses;
wherein the mirror is configured to rotate; and
wherein the ultrasonic transducer and the mirror are positioned in a probe head.

2. The device of claim 1, wherein the mirror is configured to reflect the ultrasonic pulses while rotating, and wherein received ultrasonic pulses are used to determine an ultrasonic scan of material adjacent to the lens.

3. The device of claim 1, wherein a first facet of the mirror is configured to reflect the ultrasonic pulses from the ultrasonic transducer during a first range of rotation of the mirror and a second facet of the mirror is configured to reflect the ultrasonic pulses from the ultrasonic transducer during a second range of rotation of the mirror.

4. The device of claim 3, wherein the device is configured such that ultrasonic pulses received at the transducer reflected from the first facet are used to determine a first ultrasonic scan of material adjacent to the lens, and wherein the ultrasonic pulses received at the transducer reflected from the second facet are used to determine a second ultrasonic scan of material adjacent to the lens.

6. The device of claim 4, wherein device is configured such that the first and second ultrasonic scans determined from one rotation of the mirror increase a frame rate of a resultant ultrasonic image without diminishing quality of the resultant ultrasonic image.

7. The device of claim 4, wherein the device is configured such that the first and second ultrasonic scans determined from one rotation of the mirror increase the image quality of a resultant ultrasonic image without diminishing a frame rate of the resultant ultrasonic image.

8. The device of claim 1, wherein the probe head further comprises a lens configured to direct the ultrasonic pulses.

9. The device of claim 1, wherein the mirror is configured to rotate at a rotational speed of the motor.

10. The device of claim 1, wherein the ultrasonic transducer is a circular transducer with rings for beam forming.

11. The device of claim 1, wherein the mirror has at least one additional facet.

12. The device of claim 1, wherein the mirror is configured in a pyramid shape.

13. The device of claim 12, wherein the mirror is configured in a double-pyramid shape, wherein a first pyramid has at least two facets and a second pyramid has at least two facets, and wherein a base of the first pyramid is attached to a base of the second pyramid.

14. The device of claim 13, further comprising a second ultrasonic transducer disposed in the probe head on the opposing side of the mirror from the first ultrasonic transducer.

15. The device of claim 14, wherein the ultrasonic transducer is configured to transmit ultrasonic pulses at a first frequency and the second ultrasonic transducer is configured to transmit ultrasonic pulses at a second frequency that is different than the first frequency.

16. The device of claim 15, wherein the device is configured such that the ultrasonic pulses received at the first or the second transducer from the transmission of pulses at a first frequency are combined with the ultrasonic pulses received at the first or second transducer from the transmission of pulses at a second frequency to determine an ultrasonic scan of material adjacent to the lens.

17. The device of claim 15, wherein the first frequency is in the range of 1 to 5 MHz and the second frequency is in the range of 8 to 20 MHz.

18. The device of claim 1, further comprising an electronics chamber that comprises a battery and electrical circuitry, wherein the electrical circuitry is electrically connected to the ultrasonic transducer.

19. The device of claim 18, wherein the electrical circuitry is configured to control an operational parameter of the device or perform a function on the received ultrasonic pulses.

20. The device of claim 19, wherein controlling the operational parameter of the device comprises controlling at least one operational parameter selected from the group consisting of: (a) the frequency of pulses generated by the transducer, (b) the pulse length of pulses generated by the transducer, (c) the timing of pulses generated by the transducer, and (d) the number of pulses generated by the transducer; and
wherein the function performed on the received ultrasonic pulses is a function selected from the group consisting of: (a) registering the voltage variations on the transducer, (b) converting the signal to digital, (c) performing beam forming upon reception of the signals, (d) performing scan conversion, (e) performing demodulation, and (f) performing variable gain compensation.
